(19) United States
(12) Patent Application Publication
(10) Pub. No.: US 2018/0295275 A1
(43) Pub. Date: Oct. 11, 2018

(54) REMOTE IMAGING SYSTEM USER INTERFACE

(71) Applicant: Analogic Canada Corp., Saint-Laurent (CA)

(72) Inventors: Reza Zahiri AZAR, Burnaby (CA); Urvi VYAS, Cambridge, MA (US); Hani ESKANDARI, Vancouver (CA)

(73) Assignee: Analogic Canada Corporation, Saint-Laurent (CA)

(21) Appl. No.: 15/479,731

(22) Filed: Apr. 5, 2017

(51) Int. Cl. H04N 5/232; H04N 7/18; A61B 8/00; A61B 8/08

(52) U.S. Cl. CPC H04N 5/23216; H04N 7/183; H04N 5/23203; A61B 8/4405; A61B 8/461; A61B 8/5207; A61B 8/467; H04N 5/23293

(57) ABSTRACT

A system includes an imaging system configured to generate an image during an imaging procedure in an examination room and a computing system located remote from the examination room. The imaging system further includes a communication interface configured to transmit the image to the computing system. The computing system further includes a complimentary communication interface configured to receive the image and a processor configured to transmit a feedback signal, which is determined based on the image, to the imaging system. The imaging system receives the signal via the communication interface, and further includes a controller that sets at least one value of one from a group of: a scanning parameter, or a visualization parameter of the imaging system, based on the signal.

### TECHNICAL FIELD

The following generally relates to imaging and more particularly to a remote user interface for imaging and is described with particular application to ultrasound imaging; however, the following is also amenable to other imaging modalities.

### BACKGROUND

Ultrasound imaging provides information about interior characteristics of an object or subject. An ultrasound imaging scanner has included at least a transducer array with one or more transducing elements excitable to transmit an ultrasound signal (e.g., a pressure wave) into the object or subject. As the signal traverses (static and/or moving) structure therein, portions of the signal are attenuated, scattered, and/or reflected off the structure, with some of the reflections (echoes or echo signals) traversing back to the one or more elements.

The one or more elements receive the echoes and convert them into electrical signals indicative thereof. The electrical signals are processed to generate one or more images of the interior characteristics of the object or subject, which can be displayed via a monitor display. In B-mode ultrasound imaging, the received echoes correspond to a two-dimensional (2-D) slice from the face of the transducer array through the object or subject, and the image is a 2-D image of that slice. Other modes include A-mode, C-plane, Doppler, 3-D, 4-D, etc.

Under certain instances, e.g., in emergency and/or surgical rooms, an ultrasound imaging scanner is used to produce an image(s) used to make a quick clinical decision. In some circumstances, the clinician making the quick clinical decision is not a sonographer trained in the use of the ultrasound imaging scanner and/or may not know how to optimize the scanning and/or visualization parameters. In such circumstances, it could be helpful to have a trained sonographer to aid in the clinician with operating the ultrasound imaging scanner.

### SUMMARY

Aspects of the application address the above matters, and others.

In one aspect, a system includes an imaging system configured to generate an image during an imaging procedure in an examination room and a computing system located remote from the examination room. The imaging system further includes a communication interface configured to transmit the image to the computing system. The computing system further includes a complimentary communication interface configured to receive the image and a processor configured to transmit a feedback signal, which is determined based on the image, to the imaging system. The imaging system receives the signal via the communication interface, and further includes a controller that sets at least one value of one from a group of: a scanning parameter or a visualization parameter of the imaging system, based on the signal.

In another aspect, a method includes generating, with an imaging system in an examination room and during an imaging procedure, ultrasound images. The method further includes transmitting, with the imaging system, the ultrasound images to a computing system located external to the examination room. The method further includes receiving, with the imaging system, feedback from the computing system, wherein the feedback is generated based on the ultrasound images. The method further includes setting, with the imaging system, at least one value of one from a group of: a scanning parameter and a visualization parameter of the imaging system, based on the feedback.

In another aspect, a method includes receiving, with a computing system remote from an examination room, an ultrasound image generated by an imaging system during an imaging procedure. The method further includes generating, with the computing system, a signal that controls an aspect of the imaging system during the imaging procedure. The method further includes transmitting, with the computing system, the signal to the imaging system, wherein the imaging system sets at least one value of one from a group of: a scanning parameter and a visualization parameter of the imaging system, based on the signal.

Those skilled in the art will recognize still other aspects of the present application upon reading and understanding the attached description.

### BRIEF DESCRIPTION OF THE DRAWINGS

The application is illustrated by way of example and not limited by the figures of the accompanying drawings, in which like references indicate similar elements and in which:

FIG. 1 schematically illustrates an example system which includes an ultrasound imaging system and at least one computing system, in accordance with an embodiment described herein;

FIG. 2 schematically illustrates an example of the ultrasound imaging system, in accordance with an embodiment described herein;

FIG. 3 schematically illustrates an example of the at least one computing system, in accordance with an embodiment described herein;

FIG. 4 schematically illustrates an example of the ultrasound imaging system, in accordance with an embodiment described herein;

FIG. 5 schematically illustrates an example of the at least one computing system, in accordance with an embodiment described herein;

FIG. 6 schematically illustrates another example of the at least one computing system, in accordance with an embodiment described herein;

FIG. 7 schematically illustrates another example of the at least one computing system, in accordance with an embodiment described herein;

FIG. 8 schematically illustrates another example of the at least one computing system, in accordance with an embodiment described herein;

FIG. 9 schematically illustrates another example of the at least one computing system, in accordance with an embodiment described herein;

FIG. 10 schematically illustrates an example display of the at least one computing system, in accordance with an embodiment described herein;

FIG. 11 schematically illustrates another example display of the at least one computing system, in accordance with an embodiment described herein;

FIG. 12 schematically illustrates another example display of the at least one computing system, in accordance with an embodiment described herein;

FIG. 13 illustrates an example method in accordance with an embodiment described herein;

FIG. 14 illustrates another example method in accordance with an embodiment described herein;

FIG. 15 illustrates another example method in accordance with an embodiment described herein;

FIG. 16 illustrates another example method in accordance with an embodiment described herein;

FIG. 17 illustrates another example method in accordance with an embodiment described herein;

FIG. 18 illustrates another example method in accordance with an embodiment described herein;

FIG. 19 illustrates another example method in accordance with an embodiment described herein; and

FIG. 20 illustrates another example method in accordance with an embodiment described herein.

### DETAILED DESCRIPTION

A remote user interface for imaging is described herein. The remote user interface can be employed with imaging systems such as an ultrasound imaging system, x-ray imaging, magnetic resonance imaging, positron emission imaging, single photon emission computed tomography, computed tomography and/or other imaging modality. For sake of brevity and explanatory purposes, the following example is described in relation to an ultrasound imaging system. However, it is to be understood that the remote user interface described herein is not limited to an ultrasound imaging system.

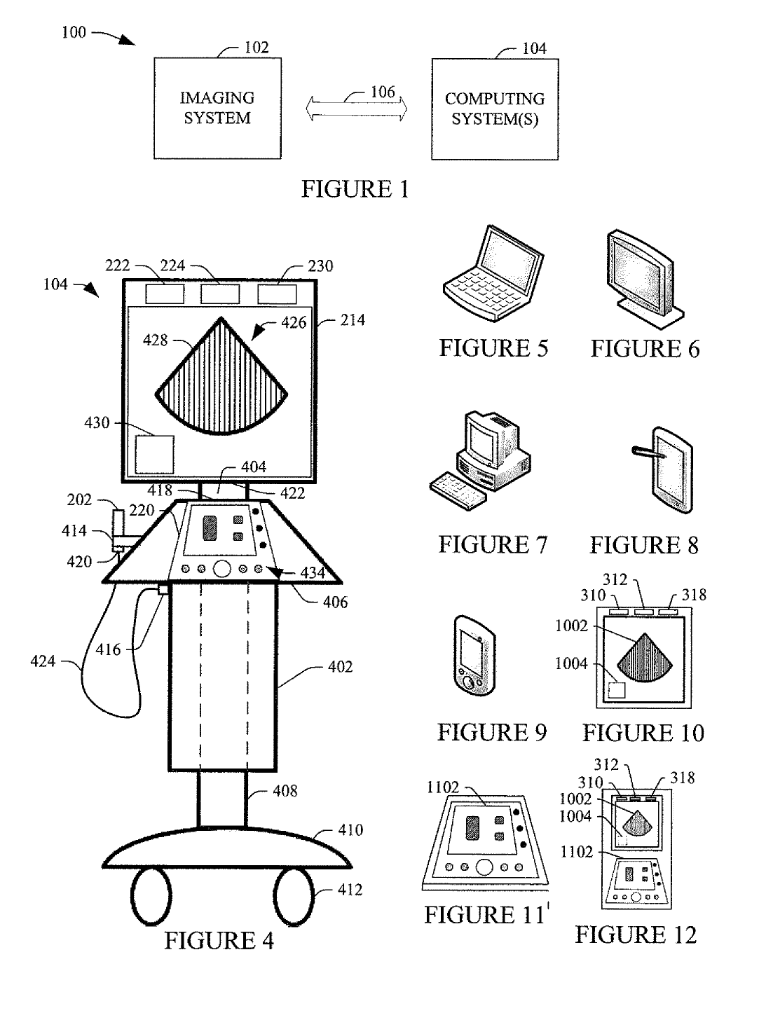

FIG. 1 schematically illustrates an example system 100 including an imaging system 102, such as an ultrasound imaging scanner or an imaging system including an ultrasound imaging system, and one or more computing systems 104. The imaging system 102 and the one or more computing systems 104 are configured to communicate via a communication path(s) 106 over a wired connection(s) (e.g., coaxial cable, twisted pair, optical fiber, universal serial bus, FireWire, etc.) and/or wirelessly (e.g., radio frequency, cellular, Bluetooth, infrared, etc.). In one instance, at least one of the one or more computing systems 104 is external to (or not in) the same room as the imaging system 102 is in when the imaging system 102 is imaging during the imaging procedure. In another instance, at least one of the one or more computing systems 104 is in the same room as the imaging system 102 when the imaging system 102 is imaging during an imaging procedure.

As described in greater detail below, in one instance the communication includes streaming a live ultrasound image(s) in real-time (e.g., as an image(s) is generated as echoes are received and processed to generate the image(s)) from the imaging system 102 to the one or more computing systems 104. Alternatively or additionally, the imaging system 102 can transmit a stored ultrasound image(s) to the one or more computing systems 104. Alternatively or additionally, the imaging system 102 and/or the one or more computing systems 104 is configured to set a value of a scanning and/or a visualization parameter(s) of the imaging system 102. Where the one or more computing systems 104 can set the value of the scanning and/or visualization parameter(s), the one or more computing systems 104 can transmit the value of the scanning and/or the visualization parameter(s) over the communication path 106.

Alternatively or additionally, the imaging system 102 is configured to record video of the imaging system 102 performing the imaging procedure and transmit the video to the one or more computing systems 104 over the communication path 106. Alternatively or additionally, the imaging system 102 is configured to record audio when the imaging system 102 is performing the imaging procedure and transmit the audio to the one or more computing systems 104 over the communication path 106. Alternatively or additionally, the one or more computing systems 104 is configured to record video and transmit the video to the imaging system 102 over the communication path 106. Alternatively or additionally, the one or more computing systems 104 is configured to record audio and transmit the audio to imaging system 102 over the communication path 106. The system receiving the video and/or audio is configured to play the video and/or audio.

In the illustrated embodiment, the imaging system 102 and/or computing system 104 also includes a software application(s) that provides video chat and/or voice call services (e.g., Skype, etc.). The imaging system 102 and/or computing system 104 can execute the software application, where the executing software application allows the imaging system 102 and/or computing system 104 to transmit and/or receive text, video and/or audible messages. Furthermore, the application may also allow the imaging system 102 and/or computing system 104 to exchange digital documents such as images, text, video, etc. Additionally or alternatively, the video chat and/or voice call services can be provided via a web service(s) (e.g., WebEx, etc.). In a variation, this feature is omitted from at least one of the imaging system 102 or the computing system 104.

Any image, video, audio and/or text shared and/or transmitted between the imagine system 102 and the computing system 104 can be “live.” With respect to imaging, the imagine system 102 can transmit data such that the display of the imaging system 102 and the display of the computing display present the same image. In one example, the image seen on the display of the imaging system 102 and on the display of the computing system 104 will be a “screen shared” image. In one instance, this does not require uploading and/or downloading a Digital Imaging and Communications in Medicine (DICOM) image(s). This “live” sharing, in one instance, also includes any annotation, measurement, and/or other information superimposed or overlaid over the image and made by either the imaging system 102 or the computing system 104.

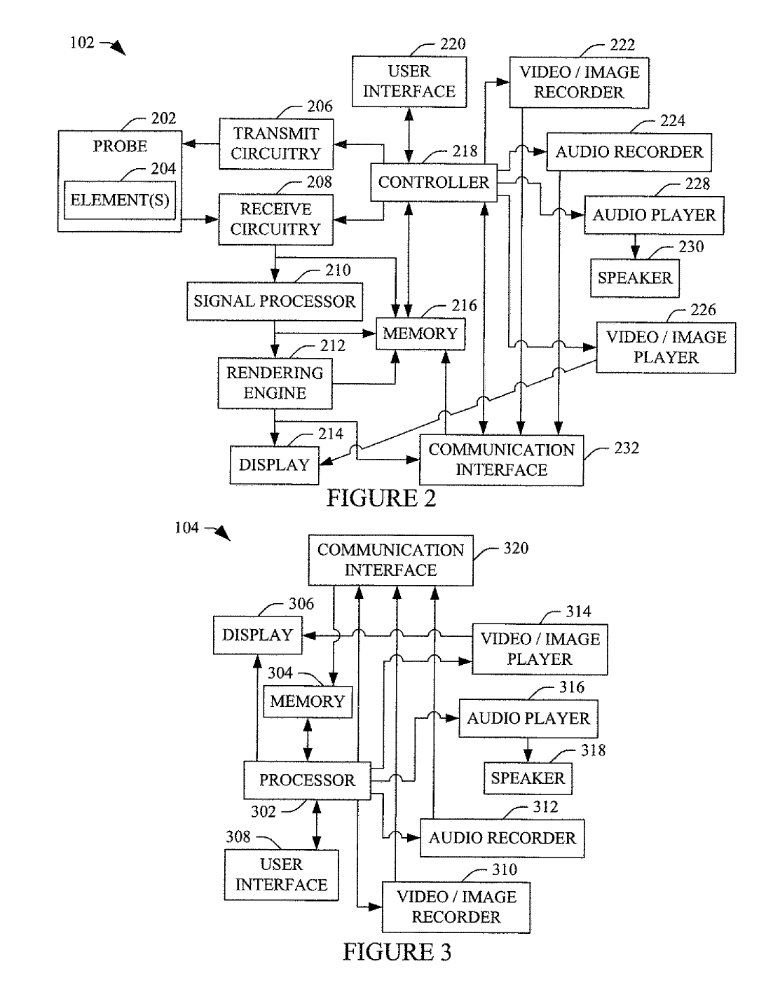

FIG. 2 schematically illustrates an example of the imaging system 102.

A probe 202 includes a one-dimensional (1-D) array or a two-dimensional (2-D) array of transducer elements 204, which are configured to transmit ultrasound signals in response to being excited, receive echo signals and generate electrical signals indicative of the received echo signals. Examples of 1-D arrays include 16, 32, 64, 128, 256, etc. arrays, and examples of 2-D arrays include 32×32, 64×64, etc. arrays, and/or other dimension arrays, including circular, elliptical, rectangular, irregular, etc. The transducer array can be linear, curved, and/or otherwise shaped, fully populated, sparse, etc.

Transmit circuitry 206 generates a set of pulses that are conveyed to the elements 204 of the transducer array. The set of pulses excites a set of the transducer elements 204 to transmit ultrasound signals. Receive circuitry 208 receives electrical signals from the elements 204 of the transducer array, which are indicative of the echoes received by elements of the transducer array. The echoes, generally, are a result of the interaction between the transmitted ultrasound signals and structure such as flowing blood cells in a vessel, organ cells, soft tissue, etc.

A signal processor 210 processes the electrical signals and produces data used to generate at least an image and/or other ultrasound information. By way of example, in one non-limiting instance, the signal processor 210 includes a beamformer, which, for B-mode imaging, is configured to apply time delays and weights to the signals and sum the weighted time-delayed signals, producing scan lines of data. The electrical signals may also be pre-processed, e.g., amplified and/or converted from analog signal to digital signals, and/or post-processed, e.g., echo-cancellation, wall-filtering, decimating, envelope detection, log-compression, FIR and/or IIR filtering, and/or other processing.

A rendering engine 212 converts the output of the signal processor 210 to generate an image(s) for display, e.g., by converting the data to the coordinate system of the display. The rendering engine 212 is also configured to process the output based on visualization parameters. For example, the rendering engine 212 can apply a pre-determined and/or user setting such as Time gain compensation (TGC) to account for tissue attenuation by increasing the received signal intensity with depth, which results in a reduction of artifacts in the uniformity of a B-mode image intensity, zoom, etc.

A display 214 is used to display the ultrasound image(s). The display 214 may be part of the imaging system 102 (e.g., integrated therein, e.g., where the imaging system include a “laptop” like console, etc.) or a separate device at least electrically connected thereto via a wired connection or wirelessly. Where the display 214 is a separate device, the display 214 can include a stand configured to rest on a surface and/or be mounted to the imaging system, a wall, etc. via a coupling device such as a bracket or the like. The display 214 can also display video received from another device such as the at least one of the one or more computing systems 104.

Memory 216 includes physical media, which is configured to store the electrical signals from the receive circuitry 208, the processed data output by the signal processor 210, and/or the scan lines from the rendering engine 212. The memory 216 is also configured to store data provided to the imaging system 102 such as video and/or audio transmitted to the imaging system 102 from another device such as the one or more computing systems 104 of FIG. 1. The memory 216 can also store other data, software applications, and/or computer readable and/or executable instructions.

A controller 218 controls one or more of the components 202-214. Such control can be based on a mode of operation (e.g., B mode, C Mode, Doppler, 3-D, 4-D, etc.) and/or otherwise. A user interface (UI) 220 includes an input device(s) (e.g., a physical button, a touch screen, a mouse, a keyboard, a keyboard, etc.) and/or an output device(s) (e.g., a touch screen, a display, etc.), which allow for interaction with the imaging system 102. With the UI 220, a user can select the mode of operation, adjust controls such as gain, focus, depth, zoom, freeze, measurement, CINE, etc., and/or add, change and/or delete an annotation, etc. overlaid over a displayed image. In one instance, the UI 220 includes a control configured to transmit (e.g., on-demand and/or automatically in response to predetermined criteria) a signal to the remote computing system 104, which invokes the remote computing system 104 to notify a user thereof (via a text and/or graphical message displayed on a monitor, an audible message, etc.) of a request for interaction therewith.

In the illustrated embodiment, the imaging system 102 further includes a video/image recorder 222, an audio recorder 224, a video/image player 226, and an audio player 228 along with a speaker 230.

In another embodiment, one or more of the components 222-228/230 are omitted. For example, with one embodiment, the imaging system 102 includes only the video/image recorder 222, only the audio recorder 224, only the video/image player 226, or only the audio player 228 and the speaker 230. In another example, the imaging system 102 includes only the video/image recorder 222 and the audio recorder 224. In another example, the imaging system 102 includes only the video/image player 226, the audio player 228 and the speaker 230. In another example, the imaging system 102 includes only the video/image recorder 222 and the video/image player 226. In another example, the imaging system 102 includes only the audio recorder 224, the audio player 228 and the speaker 230. In other examples, the imaging system 102 includes other combinations of 222-228/230.

In one instance, at least one of the components of the video/image recorder 222, the audio recorder 224, the video/image player 226, and the audio player 228/speaker 230 included with the imaging system 102 are integrated in the imaging system 102. For example, in this instance, the video/image recorder 222 can be integrated into a housing which houses the display 214 and/or other components. In another instance, at least one of the components of the video/image recorder 222, the audio recorder 224, the video/image player 226, and the audio player 228/speaker 230 included with the imaging system 102 are separate and distinct components from the imaging system 102. For example, the video/image recorder 222 can be a camera with an electrical interface to the imaging system 102.

A communication interface 232 is configured with the mechanical and/or electrical components for the imaging system 102 to transmit an image(s) (e.g., in real-time), a value of the scanning and/or a visualization parameter(s), video, audio, etc. via the communication path 106 (FIG. 1) to the at least one of the one or more computing systems 104. Alternatively or additionally, the communication interface 232 is configured with the mechanical and/or electrical components for the imaging system 102 to receive an image(s), a value of the scanning and/or a visualization parameter(s), video, audio, etc. via the communication path 106 from the at least one of the one or more computing systems 104.

The video/image recorder 222 can be used to record an ultrasound imaging procedure performed with the imaging system 104. This may include recording the positioning of the transducer array with respect to the scanned subject or object. Alternatively or additionally, this may also include recording the display 214 and/or the UI 220 when the display 214 and/or the UI 220 are being used to enter or set a value of the scanning and/or visualization parameter(s). In some embodiments, this may also include another device in the examination room. In some embodiments, this may also include a person in the examination room.

The video/image player 226 can be used to play back video recorded by the video/image recorder 222 and/or another video/image recorder such as a video/image recorder of the one or more computing systems 104. The audio recorder 224 can be used to record a person speaking in the examination room during the ultrasound imaging procedure performed with the imaging system 104. The audio player 228 can be used to play back audio via the speaker 230 such as audio recorded by the audio recorder 224 and/or another audio recorder such as an audio recorder of the one or more computing systems 104.

An example of an imaging system with a camera configured to provide a visual on the scanning environment and the scanning technique is described in U.S. patent application 2012/0179039 A1 to Pelissier et al., entitled “Methods and apparatus for producing video records of use of medical ultrasound imaging systems,” and filed on Jan. 6, 2012, which is incorporated herein in its entirety by reference. Other examples are also contemplated herein.

FIG. 3 schematically illustrates an example of the at least one of the one or more computing systems 104.

The system(s) 104 includes a processor (e.g., a central processing unit, a microprocessor, etc.) configured to execute a computer readable instruction(s) embedded or stored on memory 304, which is non-transitory computer readable medium (which excludes transitory computer readable medium), such as physical memory.

A display 306 is configured to display at least an image(s), e.g., such as an image received from the imaging system 104. In some embodiments, the display 306 is also configured to display video such as video produced by the video/image recorder 222 of FIG. 2 and/or another video/image recorder. The display 306 may be part of the at least one of the one or more computing systems 104 (e.g., integrated therein, etc.) or a separate device at least electrically connected thereto via a wired connection or wirelessly.

A user interface (UI) 308 includes an input device(s) (e.g., a physical button, a touch screen, a mouse, a keyboard, a keyboard, etc.) and/or an output device(s) (e.g., a touch screen, a display, etc.), which allow for interaction with the at least one of the one or more computing systems 104. With the UI 308, a user can suggest or adjust controls of the imaging system 102, and/or add, change and/or delete an annotation, etc. overlaid over a displayed image. In one instance, the UI 308 includes a control configured to transmit (e.g., on-demand and/or automatically in response to predetermined criteria) a signal to the imaging system 102, which invokes the imaging system 102 to notify a user thereof (via a text and/or graphical message displayed on a monitor, an audible message, etc.) of a request for interaction therewith.

In this embodiment, the at least one of the one or more computing systems 104 further includes a video/image recorder 310, an audio recorder 312, a video/image player 314, and an audio player 316 along with a speaker 318. In another embodiment, one or more of the components 310-316/318 are omitted. For example, with one embodiment, the one or more computing systems 104 includes only the video/image recorder 310, only the audio recorder 312, only the video/image player 314, or only the audio player 316 and the speaker 318.

In another example, the one or more computing systems 104 includes only the video/image recorder 310 and the audio recorder 312. In another example, the one or more computing systems 104 includes only the video/image player 314, the audio player 316 and the speaker 318. In another example, the one or more computing systems 104 includes only the video/image recorder 310 and the video/image player 314. In another example, the one or more computing systems 104 includes only the audio recorder 312, the audio player 316 and the speaker 318. In other examples, the one or more computing systems 104 includes other combinations of 310-316/318.

In one instance, at least one of the components of the video/image recorder 310, the audio recorder 312, the video/image player 314, and the audio player 316/speaker 318 included with the one or more computing systems 104 are integrated therein. For example, in this instance, the video/image recorder 310 can be integrated in the housing which houses the display 306 and/or elsewhere. In another instance, at least one of the components of the video/image recorder 310, the audio recorder 312, the video/image player 314, and the audio player 316/speaker 318 included with the imaging system 102 are separate and distinct components from the one or more computing systems 104. For example, the video/image recorder 310 can be a camera with an electrical interface to the one or more computing systems 104.

A communication interface 320 (which is complimentary to the interface 232) is configured with the mechanical and/or electrical components for the at least one of the one or more computing systems 104 to transmit an image(s), a value of a scanning and/or a visualization parameter(s), video, audio, etc. via the communication path 106 (FIG. 1) to the imaging system 102. Alternatively or additionally, the communication interface 320 is configured with the mechanical and/or electrical components for the at least one of the one or more computing systems 104 to receive an image(s) (e.g., a live or saved image), a value of a scanning and/or a visualization parameter(s), video, audio, etc. via the communication path 106 from the imaging system 102.

The video/image recorder 310 can be used to record instructions on how to use the imaging system 104. This may include instructions for positioning of the transducer array with respect to the scanned subject or object. Alternatively or additionally, this may also include the display 306 and/or the UI 308 when the display 306 and/or the UI 308 are being used to enter or set a value of a scanning and/or visualization parameter(s).

The audio recorder 312 can be used to record a person providing instructions for using the imaging system 104. The video/image player 314 can be used to play back video recorded by the video/image recorder 310 and/or another video/image recorder such as the video/image recorder 222 of the imaging system 102. The audio player 316 can be used to play back audio via the speaker 318 such as audio recorded by the audio recorder 312 and/or another audio recorder such as the audio recorder 224 of the imaging system 102.

In one instance, the remote computing system 104 is configured to connect to a medical device in a HIPAA (Health Insurance Portability and Accountability Act) and PCI (Payment Card Industry) compliant manner A non-limiting example of suitable software is “TeamViewer,” a product of a TeamViewer GmbH. In general, TeamViewer is a computer software package for remote control, desktop sharing, online meetings, web conferencing and file transfer between computers. For security, TeamViewer uses RSA public/private key exchange and AES (Advanced Encryption Standard) session encryption, two factor authentication and whitelisting.

FIG. 4 schematically illustrates an example of the imaging system 104.

In this example, the imaging system 104 includes a mobile mechanical support 402 with a display monitor support 404, a console support 406, a stand 408, and a base 410 with movers 412 such as one or more wheels, casters, etc. The display 214 is attached to the monitor support 404 and UI 220 is attached to the console support 404. A probe support 414 is configured to support the probe 202. The UI 220 includes a probe interface 416 and a display monitor interface 418, which respectively are complementary to the UI interfaces 420 and 422. The probe interface 416 and the UI interface 420 are in electrical communication via a cable 424 connected there between.

In the illustrated example, the display 214 at least includes a region 426 for displaying an ultrasound image 428. Where the imaging system 102 is configured to display video, e.g., received from the one or more computing systems 104, the display 214 also include a region 430 for displaying the video. The illustrated region 430 is for explanatory purposes and is not limiting. For example, the illustrated relative size, shape, location, etc. are not limiting. Furthermore, in one embodiment at least one of the size, the shape, the location, etc. are not limiting is adjustable. Furthermore, the region 426 and/or another display region can alternatively or additionally be part of the UI 220.

Where the imaging system 102 is configured to playback audio, e.g., received from the one or more computing systems 104, the display 214 also include the speaker 230 or the like for playing the audio. Where the imaging system 102 is configured to record audio, the display 214 also include the microphone 224. Where the imaging system 102 is configured to record video, the display 214 also include the video/image recorder 222. Likewise, the illustrated speaker 230, microphone 224 and/or video/image recorder 222 are for explanatory purposes and are not limiting and/or can alternatively or additionally be part of the UI 220 and/or the console support 406, or electrically connected to the imaging system 102.

As described herein, in some embodiments one or more of the region 426, speaker 230, microphone 224 and/or video/image recorder 222 are omitted. For example, in one embodiment, the imaging system 102 includes only the region 430 and not the speaker 230, microphone 224 and/or video/image recorder 222. In another embodiment, the imaging system 102 includes only the speaker 230. In another embodiment, the imaging system 102 includes both the speaker 230 and the region 430. In another embodiment, the imaging system 102 includes none of the region 430, speaker 230, microphone 224 and/or video/image recorder 222.

In a variation, the imaging system 102 is a hand-held ultrasound apparatus. Examples are described in U.S. Pat. No. 7,699,776 to Walker et al., entitled “Intuitive Ultrasonic Imaging System and Related Method Thereof,” and filed on Mar. 14, 2003, Ser. No. 13/017,344 to O'Connor, entitled “Ultrasound imaging apparatus,” and filed on Jan. 31, 2011, and U.S. Pat. No. 8,226,562 to Pelissier, entitled “Hand-Held Ultrasound System Having Sterile Enclosure,” and filed on Aug. 7, 2008, all three of which are incorporated herein in their entireties by reference.

FIG. 5-9 show examples of the one or more computing systems 104. In FIG. 5, the one or more computing systems 104 is configured as a laptop computer. In FIG. 6, the one or more computing systems 104 is configured as a touch screen computer. In FIG. 7, the one or more computing systems 104 is configured as desktop computer. In FIG. 8, the one or more computing systems 104 is configured as tablet. In FIG. 9, the one or more computing systems 104 is configured as a smartphone or personal data assistant (PDA). Other configurations are also contemplated herein.

FIG. 10-12 show examples of the display interfaces of the one or more computing systems 104 of FIG. 5-9.

In FIG. 10, the display interface shows a copy 1002 of the image 428 shown on the imaging system 102 of FIG. 4. As described herein, the image may be a live real-image image from the imaging system 102 or a previously generated image stored in memory. In one instance, the image in FIG. 10 is displayed with a graphical user interface similar to that used in FIG. 4 such that the information displayed in FIG. 10 is the same as that in FIG. 4, or clones the image display in FIG. 4. In this example, the display also includes a video display window or region 1004, the speaker 318, and the audio recorder 312 and the video/image recorder 310.

These regions, respectively, are configured to display video received from the imaging system 102, present audio received from the imaging system 102, record audio, and record video. As discussed herein, the displayed video and/or presented audio can be of a person in the examiner room such as the clinician operating the imaging system 102 to perform an imaging procedure, and the recorded video and/or audio can be instructions from remote person. Similar to the imaging system of FIG. 4, one or more, or all of, the components 1004, 318, 312 and/or 310 can be omitted.

In FIG. 11, the display interface shows a copy 1102 of the UI 220 of the imaging system 102 of the FIG. 4. The copy 1102 of the UI 220 displayed in FIG. 11 can be directly used to set a value of a scanning and/or a visualization parameter. In this instance, the set parameter is transmitted to controller 218 (FIG. 2) of the imaging system 102 via the communication interfaces 232 and 320. In FIG. 12, the display interface includes a combination of FIGS. 10 and 11, including the components 1004, 318, 312 and/or 310, and the copy 1102 of the UI 220.

One or more embodiments described herein may provide one or more of the following: Live Image Optimization Remotely; Retrospective Image Correction Remotely, and/or Education/Training. For example, in one instance, the approach described herein may allow a remote user (e.g., in the examination room abut away from the examination field, outside of the examination room but within the facility, remote from the facility, etc.) to view/optimize an ultrasound images displayed via a display monitor of the ultrasound imaging scanner being used to image the object or subject allowing for a trained technician to improve image quality.

Additionally or alternatively, the approach described herein may allow the remote user to re-optimize, re-annotate and/or complete worksheets on ultrasound images away from the ultrasound imaging scanner, which may improve workflow and increase device throughput (e.g., finishing worksheets and/or annotations without stopping the use of the ultrasound imaging scanner, which can be cleaned or used for a next subject or object. Additionally or alternatively, the approach described herein may allow a user to educate and/or train one or more remote users on the use of the ultrasound imaging scanner. In this approach, the ultrasound imaging scanner may be equipped with a camera to provide a visual on the scanning environment and the scanning technique.

The following describes examples user cases in accordance with an embodiment herein. In one example, the one or more computing systems 104 is used in an active remote viewer(s). In this example, the one or more computing systems 104 allows a viewer (or multiple viewers) to remotely connect to a live scanning session of the imaging system 102 and provide feedback.

In one instance, this includes using the system 100 for remote control. This allows the one or more computing systems 104 to control the imaging system 102 by adjusting a scanning parameter(s) on the UI 220 of the imaging system 102 remotely for a remote user. This can allow the sonographer to can control the imaging system 102 without having to go back and forth between the imaging system 102 and subject or object when an imaging adjustment is desired or needed. An example of this is shown in connection with FIG. 11.

In another instance, this includes the above along with a live stream of ultrasound images. This is an extension of the previous mode where in addition to the UI 220 control, a live stream of ultrasound images can also be offered for remote viewing purposes. This option can also be used by a remote user or sonographer. An example of this is shown in connection with FIG. 12.

In another instance, this includes using the system 100 for remote training. This allows the remote viewer to provide instruction to the sonographer on what to do. An example of this is shown in connection with FIG. 4. This can be combined with FIG. 10, 11 or 12 to provide a visual on the scanning environment and the scanning technique. The user being trained may be in the examination room with the imaging system 102, in another location with the one or more computing systems 104, or in yet another location with another device.

With surgical intraoperative ultrasound, the above allows a trained sonographer to control the image quality without having to be in the surgical “clean” field. This can be useful in surgical setups where ultrasound system is not necessarily close to the sonographer or when the ultrasound system might be held in place using a stepper or holder. The remote viewing and control facilities can be tailored in both security and usability modes that are more tailored to the human market.

In another instance, this includes using the system 100 a history session. This allows some steps of the ultrasound exam to be carried out on the one or more computing systems 104, for example post processing (re-measuring, annotating, changing gray scale, etc.) on ultrasound images after the images were acquired by the imaging system 102 can be done on the one or more computing systems 104 instead of the imaging system 102.

Annotating, measuring and/or “scrubbing” (changing post-processing parameters) on ultrasound images can be done in the one or more computing systems 104 instead of using the imaging system 102 for making such changes, as is done today allowing for the imaging system 102 to be used for further exams or allowing for a “knowledgeable” user to make such changes before the worksheet is completed. For example, in case of an emergency medicine application, after the sonographer has completed an examination with the imaging system 102, the reporting feature can be completed on the one or more computing systems 104, not next to the object or subject or the imaging system 102 but at a different location. The imaging system 102 can be used for the next patient while the reporting for the last patient is completed (or a device cleanup is performed, in case of a surgical system). Any correction to the labeling on the examination can also be completed on the history session saving time (in the emergency department or operating room) and allowing for trained users to make these changes, instead of the emergency department or operating room staff.

Another clinical utility is for emergency medicine facilities in rural areas with limited access to specialized physicians. For these facilities, a user (sonographer or on-call physician) can perform exams of a specific organ and transfer to a specialized physician who is at a different location for review. The specialized physician can use the one or more computing systems 104 to load the acquired ultrasound frames, adjust post-processing parameters, examine the resulting images, make a diagnosis and advise the emergency facility to take appropriate actions.

In another instance, this includes using the system 100 for a passive remote viewer(s). This allows single or multiple of the one or more computing systems 104 to connect to a live session as an observer. The observer can see both imaging parameters and live ultrasound images at the same time. This setup can be used for remote supervision by allowing the instructor to observe the scanning session. It can also be used for training purposes where inexperienced users can observe and learn how the scanning session is being carried out. This mode can also be used off-line by recording the session and reviewing it afterwards.

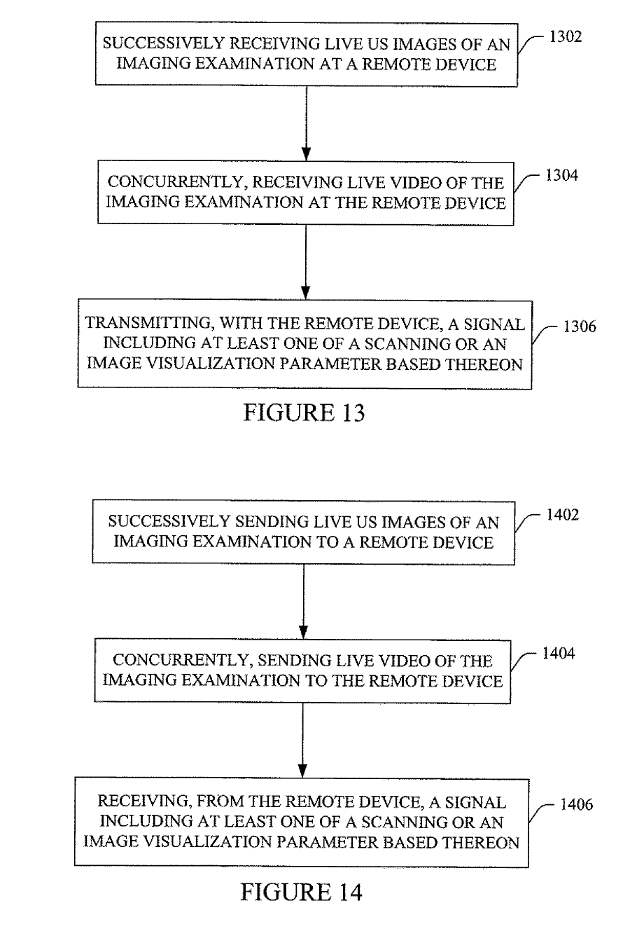

FIGS. 13-20 illustrate methods in accordance with embodiments described herein. It is to be understood that the acts in the following methods are provided for explanatory purposes and are not limiting. As such, one or more of the acts may be omitted, one or more acts may be added, one or more acts may occur in a different order (including simultaneously with another act), etc.

FIG. 13 illustrates a method in accordance with an embodiment herein.

The method includes (1302) successively receiving live ultrasound images in real-time with the one or more computing system(s) 104 and displaying the images in real-time in an image display window of the display 306 of the one or more computing system(s) 104, where each live ultrasound image is generated by the imaging system 102 in real-time during an imaging examination with the imaging system 102.

The method further includes (1304) concurrently, receiving live video of the imaging examination with the one or more computing system(s) 104 and displaying the live video in real-time in a video display window 1004 of the display 306 of the one or more computing system(s) 104, wherein the live video is generated by the video/image recorder 222 of the imaging system in real-time during the imaging examination.

The method further includes (1306) transmitting a signal including at least one of a value of a scanning or an image visualization parameter, which is determined based on at least one source from a group comprising the following sources: the received live ultrasound images and the received lived video.

FIG. 14 illustrates another method in accordance with an embodiment herein.

The method includes (1402) successively sending live ultrasound images in real-time with the imaging system 102 to the one or more computing system(s) 104 which are displayed in real-time in an image display window of the display 306 of the one or more computing system(s) 104, where each live ultrasound image is generated by the imaging system 102 in real-time during an imaging examination with the imaging system 102.

The method further includes (1404) concurrently, sending live video of the imaging examination with the imaging system 102 to the one or more computing system(s) 104 which is displayed in real-time in a video display window 1004 of the display 306 of the one or more computing system(s) 104, wherein the live video is generated by the video/image recorder 222 of the imaging system in real-time during the imaging examination.

The method further includes (1406) receiving a signal including at least one of a value of a scanning or an image visualization parameter, which is determined based on at least one source from a group comprising the following sources: the received live ultrasound images and the received lived video.

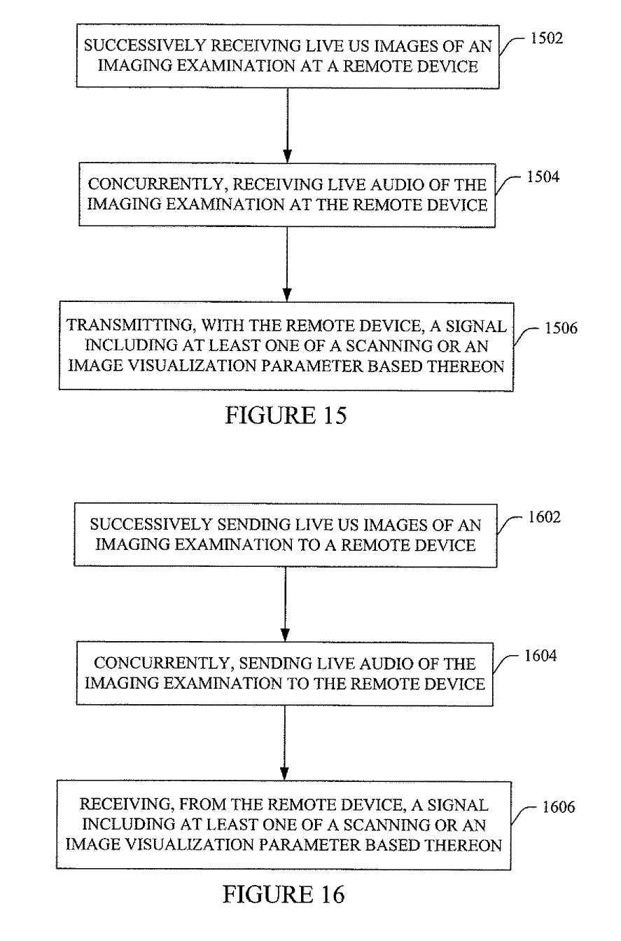

FIG. 15 illustrates another method in accordance with an embodiment herein.

The method includes (1502) successively receiving live ultrasound images in real-time with the one or more computing system(s) 104 and displaying the images in real-time in an image display window of the display 306 of the one or more computing system(s) 104, where each live ultrasound image is generated by the imaging system 102 in real-time during an imaging examination with the imaging system 102.

The method further includes (1504) concurrently, receiving live audio of the imaging examination with the one or more computing system(s) 104 and playing the live audio in real-time via an audio region 318 of the display 306 of the one or more computing system(s) 104, wherein the live audio is recorded by the audio recorder 224 of the imaging system in real-time during the imaging examination.

The method further includes (1506) transmitting a signal including at least one of a value of a scanning or an image visualization parameter, which is determined based on at least one source from a group comprising the following sources: the received live ultrasound images and the received lived audio.

FIG. 16 illustrates another method in accordance with an embodiment herein.

The method includes (1602) successively sending live ultrasound images in real-time with the imaging system 102 to the one or more computing system(s) 104 which are displayed in real-time in an image display window of the display 306 of the one or more computing system(s) 104, where each live ultrasound image is generated by the imaging system 102 in real-time during an imaging examination with the imaging system 102.

The method further includes (1604) concurrently, sending live audio of the imaging examination with the imaging system 102 to the one or more computing system(s) 104 and playing the live audio in real-time via an audio region 318 of the display 306 of the one or more computing system(s) 104, wherein the live audio is recorded by the audio recorder 224 of the imaging system in real-time during the imaging examination.

The method further includes (1606) receiving a signal including at least one of a value of a scanning or an image visualization parameter, which is determined based on at least one source from a group comprising the following sources: the received live ultrasound images and the received lived audio.

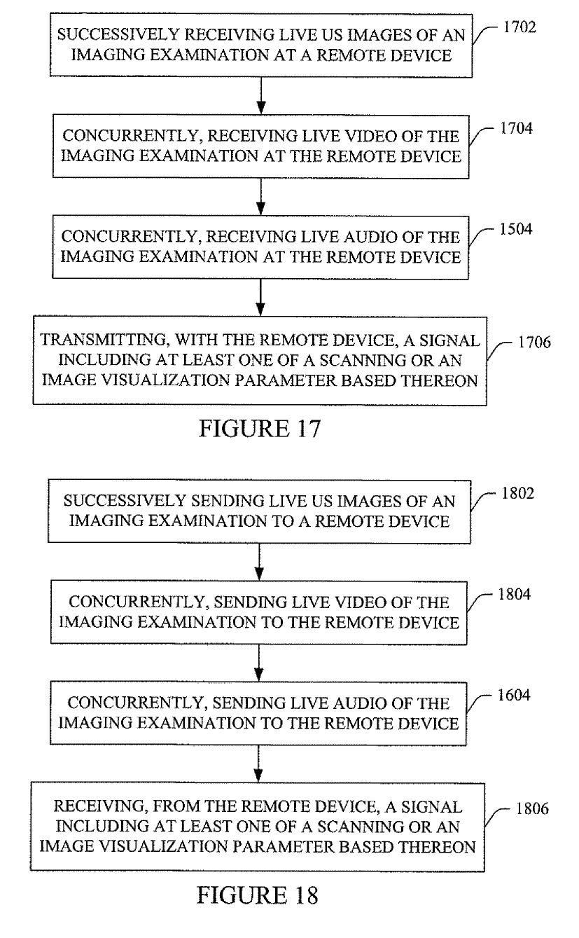

FIG. 17 illustrates another method in accordance with an embodiment herein.

The method includes (1702) successively receiving live ultrasound images in real-time with the one or more computing system(s) 104 and displaying the images in real-time in an image display window of the display 306 of the one or more computing system(s) 104, where each live ultrasound image is generated by the imaging system 102 in real-time during an imaging examination with the imaging system 102.

The method further includes (1704) concurrently, receiving live video of the imaging examination with the one or more computing system(s) 104 and displaying the live video in real-time in a video display window 1004 of the display 306 of the one or more computing system(s) 104, wherein the live video is generated by the video/image recorder 222 of the imaging system in real-time during the imaging examination.

The method further includes (1706) concurrently, receiving live audio of the imaging examination with the one or more computing system(s) 104 and playing the live audio in real-time via an audio region 318 of the display 306 of the one or more computing system(s) 104, wherein the live audio is recorded by the audio recorder 224 of the imaging system in real-time during the imaging examination.

The method further includes (1708) transmitting a signal including at least one of a value of a scanning or an image visualization parameter, which is determined based on at least one source from a group comprising the following sources: the received live ultrasound images, the received lived video, and the received lived audio.

FIG. 18 illustrates another method in accordance with an embodiment herein.

The method includes (1802) successively sending live ultrasound images in real-time with the imaging system 102 to the one or more computing system(s) 104 which are displayed in real-time in an image display window of the display 306 of the one or more computing system(s) 104, where each live ultrasound image is generated by the imaging system 102 in real-time during an imaging examination with the imaging system 102.

The method further includes (1804) concurrently, sending live video of the imaging examination with the imaging system 102 to the one or more computing system(s) 104 which is displayed in real-time in a video display window 1004 of the display 306 of the one or more computing system(s) 104, wherein the live video is generated by the video/image recorder 222 of the imaging system in real-time during the imaging examination.

The method further includes (1806) concurrently, sending live audio of the imaging examination with the imaging system 102 to the one or more computing system(s) 104 and playing the live audio in real-time via an audio region 318 of the display 306 of the one or more computing system(s) 104, wherein the live audio is recorded by the audio recorder 224 of the imaging system in real-time during the imaging examination.

The method further includes (1808) receiving a signal including at least one of a value of a scanning or an image visualization parameter, which is determined based on at least one source from a group comprising the following sources: the received live ultrasound images, the received lived video, and the received lived audio.

With FIGS. 13 and 18, in one instance, the live ultrasound images along with the audio and/or video are encoded and processed into different streams and then received and combined into a reconstituted signal and presented for evaluation at the one or more computing system(s) 104. In another instance, the live ultrasound images along with the audio and/or video are encoded in a same stream.

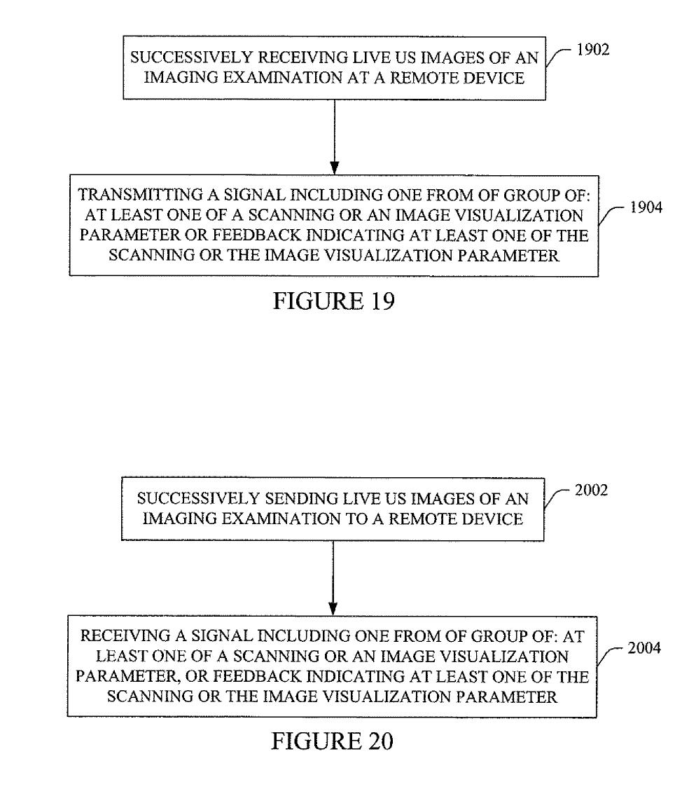

FIG. 19 illustrates another method in accordance with an embodiment herein.

The method includes (1902) successively receiving live ultrasound images in real-time with the one or more computing system(s) 104 and displaying the images in real-time in the image display window 1004 of the display 306 of the one or more computing system(s) 104, where each live ultrasound image is generated by the imaging system 102 in real-time during an imaging examination with the imaging system 102.

The method further includes (1904) transmitting a signal including one from of group of: at least one of a value of a scanning or an image visualization parameter, which is determined based on the received live ultrasound images, or feedback indicating at least one of the value of the scanning or the image visualization parameter. In one instance, the feedback is video feedback. In another instance, the feedback is audio feedback. In another instance, the feedback includes video and audio feedback. The feedback can be individual or combined streams.

FIG. 20 illustrates another method in accordance with an embodiment herein.

The method includes (20 02) successively sending live ultrasound images in real-time with the imaging system 102 to the one or more computing system(s) 104 which are displayed in real-time in the image display window 1004 of the display 306 of the one or more computing system(s) 104, where each live ultrasound image is generated by the imaging system 102 in real-time during an imaging examination with the imaging system 102.

The method further includes (2004) receiving a signal including one from of group of: at least one of a value of a scanning or an image visualization parameter, which is determined based on the received live ultrasound images, or feedback indicating at least one of the value of the scanning or the image visualization parameter. In one instance, the feedback is video feedback. In another instance, the feedback is audio feedback. In another instance, the feedback includes video and audio feedback. The feedback can be individual or combined streams.

In another embodiment, one or more of the methods of FIGS. 13-20 can be combined. For example, in one non-limiting instance at least one of method FIGS. 13-20 can be combined with at least one of the remaining methods of the methods FIGS. 13-20. In one instance, the resulting method include bi-directional communication of video and/or audio signals between the imaging system 102 and the one or more computing systems 104. In another instance, the resulting method include one-way communication of video and/or audio signals between the imaging system 102 and the one or more computing systems 104.

The methods described herein may be implemented via one or more processors (e.g., a central processing unit, a microprocessor, etc.) configured to execute a computer readable instruction(s) embedded or stored on memory 304, which is non-transitory computer readable medium (which excludes transitory computer readable medium), such as physical memory. Additionally or alternatively, the one or more processors can execute instructions carried by transitory medium such as a signal or carrier wave.

The following provides example workflow in accordance with an embodiment herein.

For connection initiation, a user of the imaging system 102 initiates contact through the UI 220 (e.g., a physical or touchscreen button) of the imaging system 102. A pop-up (email, etc.) notification is presented via the display 306 of the computing system 104. A user of the computing system 104 accepts, via the UI 308, the notification and initiates a request for screen sharing. A pop-up notification and/or password prompt is presented via the display 214 of the imaging system 102 to accept screen sharing request. The user of the imaging system 102 accepts, via the UI 220, the screen sharing request in accordance with the type of approval for which it is prompted. The computing system 104 shows a remote medical device screen in a sessions tab. The user of the computing system 104 initiates, via the UI 308, text, audio, and/or video through the sessions tab to start one way communication. The user of the imaging system 102 accepts, via the UI 220, the text, audio, and/or video, depending upon the user's choice to start two-way communication.

For the session, a user of the computing system 104 provides an instruction, a parameter, a parameter adjustment, etc. via the communication. The user of the imaging system 102 follows the instruction. The user of the imaging system 102 can request, via the communication, remote control support via audio, video and/or text. The user of the computing system 104 actuates or invokes, via the UI 308, a control for remote control. The user of the computing system 104 controls an operation of the imaging system 102. The user of the imaging system 102 confirms via the communication requirements having met via text, audio, and/or video.

For the connection termination, the user of the imaging system 102 via the communication informs the computing system 104 that the session is to be terminated. The user of the computing system 104 confirms termination via text, audio, and/or video, and logs of the remote control session. In response, the remote session tab closes, and a pup-up window provides a log out notification. Optionally, a pop up window informing end of session and asking the user to rate the service and enter any comments is presented, and the user of the imaging system 102 rates the service and/or enters comments, and closes the window. The user of the computing system 104 optionally enters notes and/or closes the window.

The application has been described with reference to various embodiments. Modifications and alterations will occur to others upon reading the application. It is intended that the invention be construed as including all such modifications and alterations, including insofar as they come within the scope of the appended claims and the equivalents thereof.

1. A system, comprising:
an ultrasound imaging system including a transducer array configured to acquire data with the acquired data based on a set of scanning parameters and generate an ultrasound image with the acquired data during an imaging procedure in an examination room; and
a computing system located remote from the examination room, wherein the imaging system further includes a communication interface configured to transmit the ultrasound image to the computing system,
wherein the computing system further includes a complimentary communication interface configured to receive the ultrasound image and a processor configured to transmit a feedback signal, which is determined based on the ultrasound image, to the imaging system, and
wherein the imaging system receives the feedback signal via the communication interface, wherein the feedback signal includes a value of a scanning parameter of interest of the scanning parameters, and wherein the imaging system further includes a controller that sets the scanning parameters of the ultrasound imaging system with the value from the feedback signal.

2. The system of claim 1, wherein the feedback signal is indicative of a value of a visualization parameter of interest of interest.

3. The system of claim 2, wherein the imaging system further comprises a video/image recorder configured to record video of the imaging system during the procedure, where the controller transmits the video to the computing system, which displays the video, and wherein the signal is further determined based on the displayed video.

4. The system of claim 2, wherein the imaging system further comprises an audio recorder configured to record audio during the procedure, where the controller transmits the audio to the computing system, which plays the audio, and wherein the signal is further determined based on the audio.

5. The system of claim 2, wherein the imaging system further comprises a video/image recorder configured to record video of the imaging system during the procedure and an audio recorder configured to record audio during the procedure, where the controller transmits the video and audio to the computing system, which displays the video plays the audio, and wherein the signal is further determined based on the displayed video and played audio.

6. The system of claim 1, wherein the computing system further comprises a video/image recorder configured to record video with an instruction for performing the imaging procedure, and the feedback signal includes the recorded video with the instruction.

7. The system of claim 1, wherein the computing system further comprises an audio recorder configured to record audio with an instruction for performing the imaging procedure, and the feedback signal includes the recorded audio with the instruction.

8. The system of claim 1, wherein the computing system further comprises a video/image recorder configured to record video with an instruction for performing the imaging procedure and an audio recorder configured to record audio with an instruction for performing the imaging procedure, and the feedback signal includes the recorded video and audio with the instruction.

9. The system of claim 1, wherein the imaging system further comprises a video/image recorder configured to record video of a transducer array of the imaging system during the procedure and an audio recorder configured to record audio during the procedure, where the controller transmits the video and audio to the computing system, which displays the video plays the audio, and
wherein the computing system further comprises a video/image recorder configured to record video with an instruction for performing the imaging procedure and an audio recorder configured to record audio with an instruction for performing the imaging procedure, where the computing system transmits the video and audio to the imaging system, which displays the video plays the audio.

10. The system of claim 1, wherein the signal processor generates and the controller transmits a live image to the computing system, and the image displayed by a first display of the computing system is a same image displayed by a second display of the imaging system.

11. The system of claim 1, wherein the imaging system and the computing system are configured to exchange at least one of live text, audio or video.

12. A method, comprising:
generating, with an imaging system in an examination room and during an imaging procedure, ultrasound images;
transmitting, with the imaging system, the ultrasound images to a computing system located external to the examination room;
receiving, with the imaging system, feedback from the computing system, wherein the feedback is generated based on the ultrasound images; and
setting, with the imaging system, at least a spatial orientation of a transducer of the imaging system, based on the feedback.

13. The method of claim 12, wherein the feedback signal is indicative of a value the spatial orientation.

14. The method of claim 13, further comprising:
transmitting, with the imaging system, at least one of video of a transducer array during the imaging procedure or audio of the imaging procedure, wherein the feedback is determined based on at least one of the video or the audio.

15. The method of claim 14, further comprising:
transmitting, with the imaging system, the at least one of video of the transducer array during the imaging procedure or the audio of the imaging procedure to at least one other computing system located external to the examination room.

16. The method of claim 12, wherein the feedback includes at least one of video with an instruction for performing the imaging procedure or audio with an instruction for performing the imaging procedure.

17. A method, comprising:
receiving, with a computing system remote from an examination room, an ultrasound image generated by an imaging system during an imaging procedure;
generating, with the computing system, a signal that controls an aspect of the imaging system during the imaging procedure; and
transmitting, with the computing system, the signal to the imaging system, wherein the imaging system sets at least a spatial orientation of a transducer of the imaging system, based on the signal.

18. The method of claim 17, wherein the feedback signal is indicative of a value of the spatial orientation.

19. The method of claim 18, further comprising:
receiving, with the computing system, at least one of video of a transducer array during the imaging procedure or audio of the imaging procedure; and
determining the signal based on at least one of the video or the audio.

20. The method of claim 17, wherein the feedback includes at least one of video with an instruction for performing the imaging procedure or audio with an instruction for performing the imaging procedure.
